(19) United States
(12) Patent Application Publication
(10) Pub. No.: US 2010/0260858 A1
(43) Pub. Date: Oct. 14, 2010

(54) DRUG DELIVERY COMPOSITION

(75) Inventors: Stephen B. Ruddy, Schwenksville, PA (US); Simon L. McGurk, Collegeville, PA (US); Rakesh Patel, Bensalem, PA (US); John Bullock, West Chester, PA (US); Raj Kewalramani, Collegeville, PA (US)

Correspondence Address: Fox Rothschild, LLP;Elan Pharma International Limited, 997 Lenox Drive, Bldg. #3, Lawrenceville, NJ, 08648, US

(73) Assignee: ELAN PHARMA INTERNATIONAL LIMITED, Athlone (IE)

(21) Appl. No.: 12/757,005

(22) Filed: Apr. 8, 2010

Related U.S. Application Data
(60) Provisional application No. 61/168,040, filed on Apr. 9, 2009.

(51) Int. Cl. A61K 9/14; A61K 38/43; A61K 31/5513; A61K 31/436; A61K 31/403; A61K 31/167; A61P 43/00

(52) U.S. Cl. 424/492; 424/490; 424/491; 424/494; 424/497; 424/94.1; 514/220; 514/291; 514/411; 514/616; 977/773

(57) ABSTRACT

A composition for delivery of a drug is disclosed. The composition has a semipermeable coating, particles of a medicament having an effective average particle size of less than or about 2 μm and at least one surface stabilizer adsorbed on the surface of the medicament particles, and a solubilizing agent.

### CROSS-REFERENCE TO RELATED APPLICATION

This application claims benefit under 35 U.S.C. §119(e) of U.S. Provisional Application No. 61/168,040, filed Apr. 9, 2009, the disclosure of which is hereby incorporated by reference herein, in its entirety.

### BACKGROUND OF THE INVENTION

Some types of oral drug delivery compositions can be described as extended-release, controlled-release, or sustained release compositions. These terms, however, have not been used consistently in the art. A more consistent term to describe these compositions collectively is “modified-release” compositions. Modified-release compositions can be defined as “compositions for which the drug release characteristics of time course and/or location are chosen to accomplish therapeutic or convenience objectives not offered by conventional dosage forms.”

In general, modified-release compositions intended for oral administration utilize drug delivery technologies to release drug over a number of hours—constantly, intermittently, or after a lag time upon ingestion. Such effects can be achieved, for example, through use of a drug release retardant contained within a matrix core or alternatively, a release-modifying film coating that envelops a core. Examples of release-modifying film coatings include those responsive to changes in pH within the environment of the GI tract (e.g., enteric coatings), or microporous coatings that govern drug release upon formation of concentration gradients or artificially created osmotic gradients.

Exemplary modified-release compositions incorporating a release-modifying film coating and/or an enteric coating include the Elan Pharma International Ltd., SODAS® (Spheroidal Oral Drug Absorption System) multiparticulate drug delivery system as exemplified in U.S. Pat. No. 6,228,398, herein incorporated by reference.

Exemplary compositions utilizing an artificially created osmotic gradient to deliver active agents include the Alza Corporation OROS® Push Pull™ osmotic drug delivery system which is described in U.S. Pat. Nos. 5,413,572; 5,324,280; and 6,419,952, each of which is incorporated by reference herein and each of which is directed to an osmotic system for delivering a beneficial agent to an environment of use. The osmotic system described therein comprises (a) an outside semipermeable wall, (b) a middle osmotically active layer, (c) a capsule comprising a beneficial agent, and (d) a passageway for dispensing the beneficial agent from the osmotic system. Another osmotic dosage form is taught in U.S. Pat. No. 4,971,790 (incorporated by reference herein), which is directed to a composition comprising a drug, a neutral hydrogel and an ionic hydrogel.

There is still, however, a need in the art to delivery poorly water-soluble drugs exhibiting low native solubility in the fluid of the environment of use.

### SUMMARY OF THE INVENTION

A drug delivery composition having a semipermeable coating, particles of a medicament, and an agent that solubilizes the medicament is provided. The medicament particles have an effective average particle size of less than or about 2 μm and a surface stabilizer adsorbed on the surface of the medicament particles.

In an embodiment, the medicament is a compound that has low native solubility in the fluid of the environment of use.

In another embodiment, the solubilizing agent is of a type and present in an amount sufficient to dissolve the medicament particles within the composition prior to delivery of the medicament to the environment of use.

In another embodiment, the solubilizing agent is a surface-active agent or a pH-modulating agent.

In another embodiment, the semipermeable coating substantially prevents the passage of medicament particles out of the drug delivery composition, but allows passage of dissolved medicament.

In another embodiment, the semipermeable coating is a controlled-porosity microporous coating comprising a poorly water-soluble or water-insoluble polymers and a water-soluble pore forming additives.

In another embodiment, the polymer of the controlled-porosity microporous coating is selected from the group consisting of cellulosic polymers such as ethylcellulose and cellulose acetate, methacrylates and phthalates, and the pore forming additive is selected from the group consisting of HPMC, PVP, and polyhydric alcohols such as mannitol, xylitol and sorbitol, and sugars such as sucrose.

In an embodiment, the drug delivery composition is in a dosage form of a capsule comprising multiparticlate beads, each bead comprises multiple layers, and, when described starting at the center of the bead and moving radially outward, has a center comprising an inert core, a layer of solubilizing agent, a layer of medicament particles having an effective average particle size of less than or about 2 μm and a surface stabilizer adsorbed on the surface of the medicament particles, and a semipermeable coating.

According to an embodiment of the invention, the composition comprises a multiparticulate pharmaceutical dosage form comprising a plurality of beads. Each bead comprising an inert substrate, a surface-active agent layer disposed about the inert substrate, and a semipermeable coating. Disposed between the surface-active agent layer and the semipermeable coating are medicament particles. The medicament particles have an effective average particle size of less than or about 2 μm and a surface stabilizer adsorbed on the surface of the particles.

In another embodiment, the medicament is a compound of Class II or Class IV (identified by the BCS (Biopharmaceutical Classification System)), which includes, but is not limited to, compounds such as tacrolimus, sirolimus, fenofibrate, carvedilol, celecoxib, and naproxen.

In another embodiment, the medicament is a weakly basic compound such as clozapine.

Yet another embodiment of the invention comprises a multiparticulate pharmaceutical dosage form comprising beads, each bead having a core of an inert substrate, a layer of medicament particles having an effective average particle size of less than or about 2 μm and a surface stabilizer adsorbed on the surface of the particles, and a semipermeable coating. Disposed between the medicament layer and the semipermeable coating is pH-modulating agent layer.

In another embodiment, the pH-modulating agent layer comprises one organic acid, possibly two or more.

In another embodiment, the organic acid is selected from the group consisting of adipic acid, ascorbic acid, citric acid, fumaric acid, gallic acid, glutaric acid, lactic acid, malic acid, maleic acid, succinic acid, tartaric acid, and other organic acids suitable for use in pharmaceutical preparations for oral administration.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is an illustration of a bead, an exemplary dosage form of the drug delivery composition of the present invention;

FIG. 2 is an illustration of the principle of operation of the bead depicted in FIG. 1;

FIG. 3 is a comparison plot of the percentage of a neutral drug dissolved over time for Composition A (an embodiment of the invention) that included a surface-active agent and a Composition C that did not (not an embodiment of the invention);

FIG. 4 is a plot of the mg amount of dissolved drug over time for a weakly basic compound formulated in an exemplary drug delivery composition of the invention;

FIG. 5 is a dissolution profile of an exemplary weakly basic medicament with a weak acid pH modulating agent;

FIG. 6 is a dissolution profile of an exemplary basic medicament with a weak acid pH modulating agent; and

FIG. 7 is a dissolution profile of an exemplary weak acid medicament with a weak base as a pH modulating agent.

### DETAILED DESCRIPTION OF THE INVENTION

“About” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

“Effective average particle size” means that for a given particle size value, x, 50% of the particles in the population are of a size less than x, and 50% of the particles in the population are of a size greater than x, when measured on a weight or volume basis. For example, a composition comprising particles of a medicament having an “effective average particle size of 2000 nm” means that 50% of the medicament particles are smaller than 2000 nm and 50% of the medicament particles are larger than 2000 nm, when measured on a weight or volume basis.

“Nanoparticle/nanoparticulate medicament” refers to a medicament in the form of solid particles having finite mass, the population of particles being characterized by an effective average particle size of less than or about 2000 nm. A nanoparticle/nanoparticulate medicament is prepared either from non-nanoparticulate API that has been subjected to a size reduction process (a so-called “top down” process), or by a molecular deposition of the medicament (a so-called “bottom up” process). Alternatively, a nanoparticle/nanoparticulate medicament is one that is manufactured using a technique intended to result in nanoparticulates. Examples of such techniques are described in more detail below. A nanoparticle/nanoparticulate medicament is distinguished from a non-nanoparticulate API, which typically does not have a reduced particle size.

According to an embodiment, non-nanoparticulate API is processed to reduce its particle size to a nanoparticulate medicament. In an embodiment, the size reduction process is a milling process. The resulting milled nanoparticulate medicament is typically characterized as having a particle size distribution characterized according to their size as a list of values or as a mathematical function that defines the relative amounts of particles present, sorted according to size. The particle size distribution of the nanoparticulate medicament may be measured by any conventional particle size measuring technique well known to those skilled in the art. Such techniques include, for example, sedimentation field flow fractionation, photon correlation spectroscopy, light scattering, and disk centrifugation. An exemplary instrument utilizing light scattering measurement techniques is the Horiba LA-950 Laser Scattering Particle Size Distribution Analyzer manufactured by Horiba, Ltd. of Minami-ku Kyoto, Japan. The resulting measured particle size distribution is typically reported using the Weibull distribution or Rosin Rammler distribution as would be understood by one of ordinary skill in the art. These reporting techniques are useful for characterizing particle size distributions of materials generated by grinding, milling, precipitation, and crushing operations.

The nomenclature “D” followed by a number indicates the numbered percentile of the particle size distribution, e.g., D₅₀, is the particle size below which 50% of the particles in a particle size distribution are smaller and above which 50% of the particles are larger, when measured on a weight or volume basis. In another example, the D₉₀ of a particle size distribution is the particle size below which 90% of particles reside, and above which only 10% of the particles reside, when measured on a weight or volume basis.

“Solubility” refers to a quantity of medicament dissolved in a given quantity of environmental fluid. In the case where the addition of medicament to the environmental fluid results in no net change in the quantity of medicament dissolved, the medicament and the environmental fluid exist in a state of “equilibrium.” The resulting solubility of medicament in the environmental fluid is defined by its “equilibrium solubility.”

“Native solubility” is the equilibrium solubility of a medicament in a specific fluid environment in the absence of a solubilization aid.

“Supersaturation” refers to the solubility state of a medicament in excess of its equilibrium solubility, characterized by a solubility that is greater than that defined by the native solubility of the medicament in a given fluid environment.

“Environment of use” or “environmental fluid” or “fluid environment” is used herein to describe the physiologic or local environmental conditions to which a typical, orally administered dosage form is exposed. An environmental fluid may consist of the stomach fluids. Exemplary physiologic conditions of the stomach include a pH value typically reported between 1 and 2 in the fasted state. Another environmental fluid may be the fluids of the small intestines. The pH values of the small intestine range from about 4.7 to 7.3. The pH of the duodenum has been reported from about 4.7 to 6.5, that of the upper jejunum to range from about 6.2 to 6.7, and that of the lower jejunum from about 6.2 to 7.3.

“Therapeutically effective amount” means the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The drug delivery composition of the invention comprises a solubilizing agent, particles of a medicament, and a semipermeable coating. The drug delivery composition is intended to provide rapid solubilization of medicament particles within the interior of the drug delivery composition and enable dissolved medicament to exit the composition by osmotically facilitated convection and/or passive diffusion.

When the drug delivery composition of the invention is at the targeted site to deliver the medicament, e.g., the stomach having a pH of about 1 to 2, the medicament particles of the drug delivery composition do not substantially dissociate from the interior of the drug delivery composition and pass through the semipermeable coating because the medicament particles are poorly soluble and/or have a low native solubility in stomach acid. Rather, when the composition is at the targeted site, the fluid environment of the target site, i.e., the stomach acid fluid, penetrates the semipermeable coating and enters the interior of the drug delivery composition. The stomach acid fluid contacts the medicament particles and the solubilizing agent therein. The solubilizing agent dissolves in the stomach acid fluid. The presence of the now-dissolved solubilizing agent provides a mechanism for dissolving the (previously insoluble) medicament particles. Once dissolved in the presence of the solubilizing agent within the interior of the drug delivery composition, the solubilized medicament is transported through the semipermeable coating out of the drug delivery composition and to the targeted environment of use.

It is believed that both the particle size of the medicament and the ability of the solubilizing agent to enhance the solubility of the medicament in the environmental fluid that penetrates the drug delivery composition serve to influence the rate of medicament delivery from the composition. Without wishing to be bound to a particular theory, it is believed that the transport mechanism is an osmotically facilitated convection and/or passive diffusion gradient.

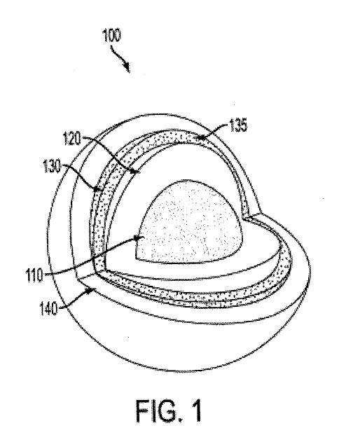

FIG. 1 illustrates an exemplary embodiment of the drug delivery composition in a bead form. In this embodiment, the drug delivery composition 100 is a multilayered bead. It would be understood by one skilled in the art that numerous beads would be placed into a capsule to create the final dosage form, a multiparticulate capsule. At the center of the bead is inert substrate 110. Surrounding inert substrate 110 is a layer of solubilizing agent 120. As shown in this embodiment, the outermost layer of the bead is semipermeable coating 140. Disposed between the layer of solubilizing agent 120 and semipermeable coating 140 is nanoparticulate medicament layer 130. The medicament particles 135 are represented by a stippling pattern for illustration purposes only.

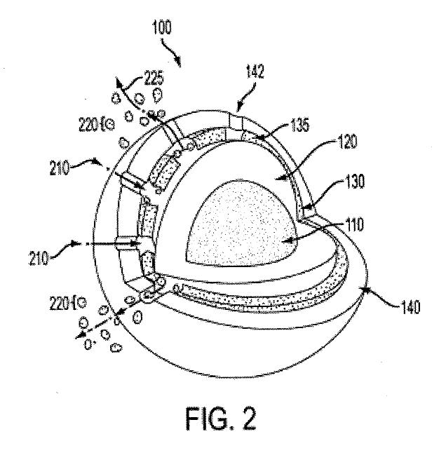

FIG. 2 is an illustration of the theoretical principle of operation of the bead depicted in FIG. 1. Without wishing to be bound to a particular theory, it is believed that the fluid 210 of the environment of use penetrates semipermeable coating 140 through pores 142. Fluid 210 passes through nanoparticulate medicament layer 130 without substantially dissolving the medicament particles 135, and contacts solubilizing agent layer 120. Solubilizing agent layer 120 is dissolved in fluid 210. The dissolved solubilizing agent assists and/or provides a mechanism for dissolving the (previously insoluble) medicament particles 135 in the fluid 210 that has penetrated composition 100. The now-solubilized medicament with solubilizing agent 220 exits the drug delivery composition 100 driven by osmotically facilitated convection and/or passive diffusion, as shown by the arrows 225.

The drug delivery composition of the present invention may be formulated into a variety of oral dosage forms. Suitable oral dosage forms include, but are not limited to, beads or pellets dispensed into capsules, granules, pills, suspensions, all tablets, or wafers. Reference to non-limiting definitions of the foregoing dosage forms may be found in the CDER Data Standards Manual (2006). According to a preferred embodiment, the present invention is a capsule containing beads or pellets.

According to the bead embodiment, the composition comprises an inert substrate, a solubilizing agent, particles of a medicament, and one or more semipermeable coatings.

In the embodiment of a bead, the center of the bead comprises an inert substrate. By “inert” it is meant that the substrate does not chemically react with the medicament in the drug delivery device. The inert substrate provides support for the solubilizing agent layer. The inert substrate may also contribute to the osmotic pressure gradient that is established across the semipermeable coating. The substrate is made from a carrier material or combinations of carrier materials. The carrier material is any soluble or insoluble, biologically acceptable material, such as sucrose or starch. Exemplary carrier materials are NON-PAREIL® seeds such as Sugar Spheres NF having a uniform diameter such as those manufactured by JRS Pharma LP, of Patterson, N.Y.

In an alternative embodiments to the bead, the inert substrate is replaced by the solubilizing agent, a combination of the solubilizing agent admixed with a binder or carrier, a medicament particles, or a combination of the medicament particles admixed with a binder or carrier.

In other dosage form embodiments, for example, the inert substrate may be eliminated altogether, for example in a compressed or matrix tablet.

The drug delivery composition comprises a solubilizing agent. The solubilizing agent is of a type and present in an amount sufficient to dissolve the medicament particles in the fluid of the environment of use. As described previously, the solubilizing agent dissolves in the fluid that has penetrated the drug delivery composition. The presence of the dissolved solubilizing agent provides a mechanism for dissolving the medicament particles (which are poorly soluble or have a low native solubility in the environmental fluid).

According to various dosage form embodiments, the solubilizing agent is admixed with a binder and forms part of the core of a bead, is a layer that is adjacent to and disposed about the inert substrate (e.g., the sugar sphere core), is a layer that is disposed between the drug layer and the semipermeable membrane, or is admixed with the other components of the composition when the dosage form is a compressed tablet or matrix tablet.

In the embodiments where the solubilizing agent is a layer that surrounds or is disposed about another layer of a bead, it is envisaged that the solubilizing agent layer may have slight defects, gaps, cracks, crevices, or holes and that there does not have to be a complete and utter surrounding.

In certain embodiments, the solubilizing agent is a surface-active agent or a pH-modulating agent.

In embodiments where the solubilizing agent is a surface-active agent, it is theorized that the mechanisms by which it dissolves the medicament is by enhancing the dissolution of the medicament particles, formation of micelles, or though formation of colloidal self-association structures. By providing a mechanism to dissolve medicaments in fluids in which the medicament would otherwise would have low native solubility, the drug delivery composition of the invention delivers to an environment of use a solution of medicament having a higher concentration than that defined by the native solubility of the medicament in the fluid environment.

Micelles are water-soluble aggregates of molecules with hydrophobic and hydrophilic portions (so-called amphiphilic molecules) which associate spontaneously. Such micelles can be in the form of small spheres, ellipsoids or long cylinders, and can also consist of bilayers with two parallel layers of amphiphilic molecules. Such bilayered micelles usually take the shape of spherical vesicles with an internal aqueous compartment. The particular surface-active agent is chosen, in part, based upon its micellular uptake ratio, which is the amount of surfactant required to dissolve a fixed amount of medicament.

Exemplary surface-active agents include, but are not limited to, ionic (e.g., anionic, cationic, and zwitterionic) and nonionic surface-active agents. Exemplary anionic (based on sulfate, sulfonate or carboxylate anions) surface-active agents include sodium dodecyl sulfate (SDS), ammonium lauryl sulfate, sodium lauryl sulfate (SLS) and other alkyl sulfate salts, sodium laureth sulfate, also known as sodium lauryl ether sulfate (SLES), alkyl benzene sulfonate, various soaps, and fatty acid salts. Exemplary cationic (based on quaternary ammonium cations) surface-active agents include cetyl trimethylammonium bromide (CTAB) a.k.a. hexadecyl trimethyl ammonium bromide, and other alkyltrimethylammonium salts, cetylpyridinium chloride (CPC), polyethoxylated tallow amine (POEA), benzalkonium chloride (BAC), and benzethonium chloride (BZT). Exemplary zwitterionic (amphoteric) surface-active agents include dodecyl betaine, dodecyl dimethylamine oxide, cocamidopropyl betaine, and coco ampho glycinate. Exemplary nonionic surface-active agents include alkyl poly(ethylene oxide), copolymers of poly(ethylene oxide) and poly(propylene oxide) [commercially called Poloxamers or Poloxamines], alkyl polyglucosides, including octyl glucoside, and decyl maltoside, fatty alcohols, including cetyl alcohol, and oleyl alcohol, cocamide MEA, cocamide DEA and polysorbates (commercially sold under the tradename Tween® by ICI Americas).

Selection of the appropriate surface-active agent is made based on a consideration of relevant medicament physicochemical properties such as the presence and type of ionizable functional groups, pka value, solubility and pH-solubility profile, salt forming characteristics, hydrophobicity, molecular size, complex formation characteristics, chemical stability, and the dose and target delivery environment for the medicament. If the medicament does not contain a functional group that is ionizable in the physiological pH range of the gastrointestinal tract, a surface-active agent is chosen based on the hydrophobicity and molecular size of the medicament and the ability of the surface-active agent to solubilize the medicament by micellerization, molecular inclusion, hydrotropy, complexation or molecular-association. If the medicament contains an ionizable functional group, additional considerations in the selection of the surface-active agent include its pH-charge-solubility profile and any charge carried by the surface-active agent. Identification of the appropriate surface-active agent can be determined using in vitro screening techniques for medicament solubility and chemical stability, which techniques are known by one of ordinary skill in the art.

The surface-active agent is present in the composition in an amount sufficient to enhance the solubility of the medicament in the environmental fluid which penetrates the composition. The surface-active agent is present in an amount from about 1%, 3%, 5%, 7%, 10%, 12%, 14%, 17%, 18%, 19%, 20%, 21%, 22%, 23%, 24%, 25%, 26%, 27%, 28%, 29%, 30%, 32%, 34%, 36%, 38%, 40%, 43%, 46%, 49%, 50% 55%, 60%, 65%, 70%, 75%, 80%, 85%, and 90% by weight of the composition. The amount of surface-active agent in the composition may also be expressed as a range between any of the above-listed individual percentages.

In embodiments where the solubilizing agent is a pH-modulating agent, it is theorized that the mechanism for dissolving the medicament particles involve modification of the pH of the fluid within the drug delivery composition. The pH-modulating agent modifies the pH of the fluid that has entered the drug delivery composition to favor the ionized form of the medicament thereby allowing the medicament (which would otherwise have a low native solubility in the fluid) to dissolve. The dissolved medicament exits the dosage form, passing through the pores of the semipermeable coating, to the environment of use in a pre-dissolved form.

Depending on the medicament, the pH-modulating agent is a weak acid or a weak base. Preferably, the pH-modulating agent is a pharmaceutically acceptable organic or inorganic weak acid or base.

In the embodiment where the pH-modulating agent is an acid, at least one organic acid, possibly two or more, are present as the pH-modulating agent. Depending on the physical and chemical properties of the medicament and the desired delivery profile, more than three pH-modulating agents are envisaged. Types of organic acids which are exemplary pH-modulating agents include, but are not limited to, adipic acid, ascorbic acid, citric acid, fumaric acid, gallic acid, glutaric acid, lactic acid, malic acid, maleic acid, succinic acid, tartaric acid, and other organic acids suitable for use in pharmaceutical preparations for oral administration such as described in WO 01/032149, herein incorporated by reference.

In the embodiment where the pH-modulating agent is a base, at least one base, possibly two or more, are present as the pH-modulating agent. Depending on the physical and chemical properties of the medicament and the desired delivery profile, more than three pH-modulating agents are envisaged. Types of bases which are exemplary pH-modulating agents include, but are not limited to, arginine, lysine, tromethamine (TRIS), meglumine, diethanolamine, triethanolamine, and conjugate bases of pharmaceutically acceptable weak acids (including those listed above), such as sodium carbonate, sodium phosphate, calcium phosphate, trisodium citrate, and sodium ascorbate.

Selection of the appropriate pH-modulating agent is made based on a consideration of relevant medicament physicochemical properties such as the number and type of ionizable functional groups (acidic or basic), pka values of the functional group(s), pH-solubility profile, salt forming characteristics, ksp, chemical stability, and the dose and target delivery environment for the medicament. For a medicament containing a weakly basic functional group the pH-modulating agent is typically an organic or inorganic weak acid possessing a pka value that is preferably at least 1 log unit lower than the pka value of the weakly basic medicament functional group. Similarly, for a medicament containing a weakly acidic functional group the pH-modulating agent is typically an organic or inorganic weak base possessing a pka value that is preferably at least 1 log unit higher than the pka value of the weakly acidic medicament functional group. If salt formation between the medicament and pH-modulating agent is possible then an agent forming a salt with a high solubility product constant (kₛₚ) is preferred.

The pH-modulating agent is present in the composition in an amount sufficient to enhance the solubility of the medicament in the environmental fluid which penetrates the composition. The pH-modulating agent is present in an amount from about 1%, 3%, 5%, 7%, 10%, 12%, 14%, 17%, 20%, 22%, 25%, 27%, 30%, 31%, 32%, 33%, 34%, 35%, 36%, 37%, 38%, 39%, 40%, 41%, 43%, 46%, 49%, 50% 55%, 60%, 65%, 70%, 75%, 80%, 85%, and 90% by weight of the composition. The amount of pH-modulating agent in the composition may also be expressed as a range between any of the above-listed individual percentages.

In certain embodiments, the composition delivers to the environment of use a solution of medicament at a concentration that is higher than that defined by the native solubility of the medicament in the same environment of use. In other words, the drug delivery composition of the invention enables the medicament to be delivered to the environment in the form of a solution that is effectively supersaturated when compared to the native solubility of the medicament in the same fluid environment.

In another embodiment, an exemplary composition of the invention delivers to the environment of use a solution of medicament at a higher concentration than a similar composition containing non-nanoparticulate API as described in the diagnostic formulation model system of Example 5.

In yet another embodiment, an exemplary composition of the invention delivers to the environment of use a solution of medicament at a higher concentration than a similar composition in the absence of a solubilizing agent as described in the diagnostic formulation model system of Example 5.

The drug delivery composition of the invention delivers dissolved medicament at a concentration that is 101%, 102%, 103%, 104%, 105%, 106%,107%,108%, 109% 100%, 110%, 120%, 130%, 140%, 150%, 160%, 170%, 180%, 190%, 200%, 210%, 220%, 230%, 240%, 250%, 260%, 270%, 280%, 290%, 300, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 700%, 800% or 1000% of the native solubility of the medicament in the environment of use, or of that achieved by a similar composition containing non-nanoparticulate API as described in the diagnostic formulation model system of Example 5, or of that achieved by a similar composition in the absence of a solubilizing agent as described in the diagnostic formulation model system of Example 5.

Alternatively stated, the drug delivery composition of the invention can deliver the medicament to the environment of use at a factor of 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, or 10.0 times the native solubility of the medicament in the environment of use, or that achieved by a similar composition containing non-nanoparticulate API as described in the diagnostic formulation model system of Example 5, or that achieved by a similar composition in the absence of a solubilizing agent as described in the diagnostic formulation model system of Example 5.

Medicaments of the invention include those compounds that are poorly water soluble. (The term “compound(s)” and “medicament(s)” are interchangeably used herein.) These compounds have solubility not greater than about 10 mg/ml in 37° C. water. In another embodiment, the compound solubility is not greater than about 1 mg/ml. In another embodiment, the compound solubility is not greater than about 0.1 mg/ml. A synonymous term to “poorly soluble” is “low aqueous solubility.” Solubility in water for many drugs can be readily determined from standard pharmaceutical reference books, for example, The Merck Index, 13th ed., 2001 (published by Merck & Co., Inc., Rahway, N.J.); the United States Pharmacopoeia, 24th ed. (USP 24), 2000; The Extra Pharmacopoeia, 29th ed., 1989 (published by Pharmaceutical Press, London); and the Physicians Desk Reference (PDR), 2005 ed. (published by Medical Economics Co., Montvale, N.J.).

Individual compounds of low solubility as defined herein include those drugs categorized as “slightly soluble,” “very slightly soluble,” “practically insoluble” and “insoluble” in USP 24, NF 19, U.S. Pharmacopeia, pp. 2254-2298.; and those drugs categorized as requiring 100 ml or more of water to dissolve 1 g of the drug, as listed in USP 24, NF 19, U.S. Pharmacopeia, pp. 2299-2304.

Compounds of the invention also include those which have low native solubility in the fluid of the environment of use. For example, the environment of use may be the gastrointestinal tract, which contains within specific regions fluids varying in pH. The pH of fasted stomach fluids is typically reported in the range of 1 to 2. The pH of small intestinal fluid is typically reported in the range of about 4.7 to 7.3. The pH of duodenal fluid has been reported in the range of about 4.7 to 6.5, those of the upper jejunum in the range of about 6.2 to 6.7, and lower jejunum, about 6.2 to 7.3. Compounds of the invention can be those medicaments that exhibit low native solublility in any one of the aforementioned environments of use, but which in another environment of use may have a high native solubility. For example, a weakly basic compound, such as clozapine, is considered to have low native solubility in a neutral pH environment, but far higher native solubility in an acidic pH environment.

Medicaments suitable for use in the invention can also be identified generally by drug class, e.g., Class II or Class IV, according to the BCS (Biopharmaceutical Classification System). Exemplary medicaments of the invention can also be identified by therapeutic class, which includes, but are not limited to, medicaments which are abortifacients, ACE inhibitors, α- and β-adrenergic agonists, α-and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents, antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, ant igonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, COX-I and COX II inhibitors, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H2 receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, NSAIDS, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors, and combinations thereof.

Further examples of suitable medicaments include, but are not limited to, acetohexamide, acetylsalicylic acid, alclofenac, allopurinol, atropine, benzthiazide, carprofen, carvedilol, celecoxib, chlordiazepoxide, chlorpromazine, clonidine, clozapine, codeine, codeine phosphate, codeine sulfate, deracoxib, diacerein, diclofenac, diltiazem, docetaxel, estradiol, etodolac, etoposide, etoricoxib, fenbufen, fenclofenac, fenprofen, fentiazac, flurbiprofen, griseofulvin, haloperidol, ibuprofen, indomethacin, indoprofen, ketoprofen, lorazepam, medroxyprogesterone acetate, megestrol, meloxicam, methoxsalen, methylprednisone, morphine, morphine sulfate, naproxen, nicergoline, nifedipine, niflumic, olanzapine, oxaprozin, oxazepam, oxyphenbutazone, paclitaxel, palperidone, phenindione, phenobarbital, piroxicam, pirprofen, prednisolone, prednisone, procaine, progesterone, pyrimethamine, risperidone, rofecoxib, asenapine, sulfadiazine, sulfamerazine, sulfisoxazole, sulindac, suprofen, tacrolimus, temazepam, tiaprofenic acid, tilomisole, tolmetic, valdecoxib, vorinostat,and ziprasidone.

Yet further exemplary medicaments include, but are not limited to, acenocoumarol, acetyldigitoxin, anethole, anileridine, benzocaine, benzonatate, betamethasone, betamethasone acetate, betamethasone valerate, bisacodyl, bromodiphenhydramine, butamben, chlorambucil, chloramphenicol, chlordiazepoxide, chlorobutanol, chlorocresol, chlorpromazine, clindamycin palmitate, clioquinol, clopidogrel, cortisone acetate, cyclizine hydrochloride, cyproheptadine hydrochloride, demeclocycline, diazepam, dibucaine, digitoxin, dihydroergotamine mesylate, dimethisterone, disulfuram, docusate calcium, dihydrogesterone, enalaprilat, ergotamine tartrate, erythromycin, erythromycin estolate, flumethasone pivalate, fluocinolone acetonide, fluorometholone, fluphenazine enanthate, flurandrenolide, guaifenesin, halazone, hydrocortisone, levothyroxine sodium, methyclothiazide, miconazole, miconazole nitrate, nitrofurazone, nitromersol, oxazepam, pentazocine, pentobarbital, primidone, quinine sulfate, stanozolol, sulconazole nitrate, sulfadimethoxine, sulfaethidole, sulfamethizole, sulfamethoxazole, sulfapyridine, tacrolimus, testosterone, triazolam, trichlormethiazide, and trioxsalen.

The amount of medicament in the composition ranges in an amount from about 10% to about 90% by weight, for example between 20% and 40%. In certain embodiments, the amount of medicament is 0.1%, 0.5%. 0.75%, 1%, 1.25%, 1.5%, 1.75%, 2%, 3%, 5%, 10%, 15%, 20%, 25%, 30%, 35%, 40%, 45%, 50%, 55%, 60%, 65%, 70%, 75%, 80%, 85%, and 90% by weight of the total composition. The amount of medicament in the composition may also be expressed as a range between any of the above-listed individual percentages.

In the exemplary embodiment dosage form of a capsule comprising beads, the beads may also include one or more isolation layers. The isolation layer serves to protect the medicament layer from the other component layers. Exemplary isolation layer components include the aqueous film coating systems sold under the Opadry® tradename by Colorcon, Inc. of West Point, Pa.

The medicament particles of the present invention have at least one surface stabilizer adsorbed on the surface thereof. Surface stabilizers useful herein physically adhere to or associate with the surface of the nanoparticulate medicament, but do not chemically react with the medicament particles. The surface stabilizers are present in an amount sufficient to substantially prevent aggregation or agglomeration of the medicament particles during formation and/or upon redispersion of the medicament particles in the environment of use. Although certain compounds suitable as surface stabilizers of the invention may also be suitable as solubilizing agents of the invention, amounts of such compounds required to function as surface stabilizers are generally insufficient to achieve substantial dissolution of the medicament particles in the fluid of the environment of use. Moreover, as defined herein, a surface stabilizer of the invention is adsorbed on the surface of the medicament particle.

Exemplary surface stabilizers include, but are not limited to, known organic and inorganic pharmaceutical excipients, as well as peptides and proteins. Such excipients include various polymers, low molecular weight oligomers, natural products, and surfactants. Useful surface stabilizers include nonionic surface stabilizers, anionic surface stabilizers, cationic surface stabilizers, and zwitterionic surface stabilizers. Combinations of more than one surface stabilizer can be used in the invention.

Representative examples of surface stabilizers include, but are not limited to, foregoing alone or in combination: hydroxypropyl methylcellulose (HPMC); dioctyl sodium sulfosuccinate (DOSS); sodium lauryl sulfate (SLS) a.k.a. sodium dodecyl sulfate (SDS); hydroxypropyl cellulose grade HPC-SL (viscosity of 2.0 to 2.9 mPa·s, aqueous 2% W/V solution, 20 DEG C, Nippon Soda Co., Ltd.); polyvinylpyrrolidone (PVP) such as Kollidone® K12 sold by BASF a.k.a. Plasdone® C-12 sold by ISP Technologies, Inc. (USA), Kollidone® K17 sold by BASF a.k.a. Plasdone® C-17 sold by ISP Technologies, Inc. (USA), Kollidone® K29/32 sold by BASF a.k.a. Plasdone® C-29/32 sold by ISP Technologies, Inc. (USA); sodium deoxycholate; block copolymers based on ethylene oxide and propylene oxide commonly known as poloxamers which are sold under the Pluronic® name by BASF (sold under the trade name Lutrol® in EU) and include Pluronic® F 68 a.k.a. poloxamer 188, Pluronic® F 108, a.k.a. poloxamer 338, Pluronic® F 127 a.k.a poloxamer 407; benzalkonium chloride a.k.a. alkyldimethylbenzylammonium chloride; copolymers of vinylpyrrolidone and vinyl acetate commonly known as copovidone sold under the tradename Plasdone® S-630 by ISP Technologies, Inc. (USA); lecithin; polyoxyethylene sorbitan fatty acid esters commonly known as polyoxyethylene 20 sorbitan monolaurate a.k.a. “polysorbate 20”, polyoxyethylene 20 sorbitan monopalmitate a.k.a. “polysorbate 40,” polyoxyethylene 20 sorbitan monooleate a.k.a. “polysorbate 80” sold under the trade names Tween® 20, Tween® 40 and Tween® 80, respectively, by ICI Americas; albumin; lysozyme; gelatin; macrogol 15 hydroxystearate sold as Solutol® 15 by BASF; tyloxapol, and polyethoxylated castor oils sold under the trade name Cremophor® EL by BASF.

Other surface stabilizers include, but are not limited to, hydroxypropylcellulose, random copolymers of vinyl pyrrolidone and vinyl acetate, casein, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000); polyethylene glycols (e.g., Carbowaxes 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.); Tetronic 1508® (T-1508) (BASF Wyandotte Corporation), Tritons X-200®, which is an alkyl aryl polyether sulfonate (Dow); Crodestas F-110®, which is a mixture of sucrose stearate and sucrose distearate (Croda Inc.); p-isononylphenoxypoly-(glycidol), also known as Olin-10G® or Surfactant 10-G®) (Olin Chemicals, Stamford, Conn.); Crodestas SL-40® (Croda, Inc.); and SA₉OHCO, which is C₁₈H₃₇CH₂C(O)N(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, and the like.

Additional examples of useful surface stabilizers include, but are not limited to, polymers, biopolymers, polysaccharides, cellulosics, alginates, phospholipids, poly-n-methylpyridinium chloride, anthryul pyridinium chloride, cationic phospholipids, chitosan, polylysine, polyvinylimidazole, polybrene, polymethylmethacrylate trimethylammonium bromide (PMMTMABr), hexyldecyltrimethylammonium bromide (HDMAB), and polyvinylpyrrolidone-2-dimethylaminoethyl methacrylate dimethyl sulfate.

Still further examples of useful stabilizers include, but are not limited to, cationic lipids, sulfonium, phosphonium, and quaternary ammonium compounds, stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy) 4 ammonium chloride or bromide, N-alkyl (C₁₂₋₁₈)dimethylbenzyl ammonium chloride, N-alkyl (C_{14 18})dimethylbenzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₂ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (sold under the ALIQUAT® 336 trade name of the Henkel Corporation), Polyquaternium-10, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quatemized polyoxyethylalkylamines, alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Additional exemplary surface stabilizers are described in detail in the Handbook of Pharmaceutical Excipients, published jointly by the American Pharmaceutical Association and The Pharmaceutical Society of Great Britain, the Pharmaceutical Press, 2005. The surface stabilizers are commercially available and/or can be prepared by techniques known in the art. Presentations of exemplary surface stabilizers are given in McCutcheon, Detergents and Emulsifiers, Allied Publishing Co., New Jersey, 2004 and Van Os, Haak and Rupert, Physico-chemical Properties of Selected Anionic, Cationic and Nonionic Surfactants, Elsevier, Amsterdam, 1993; Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990); all of which are incorporated by reference.

Exemplary methods of making compound nanoparticles are described in U.S. Pat. No. 5,145,684, the entire content of which is incorporated by reference herein. The desired effective average particle size of the invention can be obtained by controlling the process of particle size reduction, such as controlling the milling time and the amount of surface stabilizer added. Crystal growth and particle aggregation can be minimized by milling or precipitating the composition under colder temperatures, milling in the presence of or adding a surface stabilizer after size reduction, and by storing the final composition at colder temperatures.

Milling of the medicament in an aqueous solution to obtain a nanoparticulate dispersion comprises dispersing compound in water, followed by applying mechanical means in the presence of grinding media to reduce the particle size of the compound to the desired effective average particle size. The medicament can be effectively reduced in size in the presence of surface stabilizers. Alternatively, the medicament can be contacted with two or more surface stabilizers after attrition. Other compounds, such as a bulking agent, can be added to the medicament/surface stabilizer mixture during the size reduction process. Dispersions can be manufactured continuously or in a batch mode. The resultant nanoparticulate medicament dispersion can be sprayed dried and formulated into the desired dosage from.

Exemplary useful mills include low energy mills, such as a roller mill, attrition mill, vibratory mill and ball mill, and high energy mills, such as Dyno mills, Netzsch mills, DC mills, and Planetary mills. Media mills include sand mills and bead mills. In media milling, the medicament is placed into a reservoir along with a dispersion medium (for example, water) and at least two surface stabilizers. The mixture is recirculated through a chamber containing media and a rotating shaft/impeller. The rotating shaft agitates the media which subjects the compound to impacting and sheer forces, thereby reducing particle size.

Exemplary grinding media comprises media that are substantially spherical in shape, such as beads, consisting essentially of polymeric resin. In another embodiment, the grinding media comprises a core having a coating of a polymeric resin adhered thereon. Other examples of grinding media comprise essentially spherical particles comprising glass, metal oxide, or ceramic.

In general, suitable polymeric resins are chemically and physically inert, substantially free of metals, solvent, and monomers, and of sufficient hardness and friability to enable them to avoid being chipped or crushed during grinding. Suitable polymeric resins include, without limitation: crosslinked polystyrenes, such as polystyrene crosslinked with divinylbenzene; styrene copolymers, for example, PolyMill® milling media (Elan Pharma International Ltd.); polycarbonates; polyacetals, for example, Delrin® milling media (E.I. du Pont de Nemours and Co.); vinyl chloride polymers and copolymers; polyurethanes; polyamides; poly(tetrafluoroethylenes), for example, Teflon® polymers (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes. The polymer can be biodegradable. Exemplary biodegradable polymers include poly(lactides), poly(glycolide)copolymers of lactides and glycolide, polyanhydrides, poly(hydroxyethyl methacylate), poly(imino carbonates), poly(N-acylhydroxyproline)esters, poly(N-palmitoyl hydroxyproline)esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 10 μm to about 3 mm. For fine grinding, exemplary grinding media is from about 20 μm to about 2 mm. In another embodiment, exemplary grinding media is from about 30 μm to about 1 mm in size. In another embodiment, the grinding media is about 500 μm in size. The polymeric resin can have a density from about 0.8 to about 3.0 g/ml.

Another method of forming the desired nanoparticulate medicament is by microprecipitation. This is a method of preparing stable dispersions of medicaments in the presence of surface stabilizers and one or more colloid stability enhancing agents free of any trace toxic solvents or solubilized heavy metal impurities. An exemplary method comprises: (1) dissolving the compound in a suitable solvent; (2) adding the formulation from step (1) to a solution comprising at surface stabilizer to form a clear solution; and (3) precipitating the formulation from step (2) using an appropriate non-solvent. The method can be followed by removal of any formed salt, if present, by dialysis or diafiltration and concentration of the dispersion by conventional means. The resultant nanoparticulate medicament dispersion can be sprayed dried and formulated into the desired dosage from.

Another method of forming the desired nanoparticulate medicament is by homogenization. Like precipitation, this technique does not use milling media. Instead, the medicament, surface stabilizer(s) and a carrier—the “mixture” (or, in an alternative embodiment, medicament and carrier with the surface stabilizer added following reduction in particle size) constitute a process stream propelled into a process zone, which in a Microfluidizer® spray (Microfluidics Corp.) is called the Interaction Chamber. The mixture to be treated is inducted into the pump and then forced out. The priming valve of the Microfluidizer® purges air out of the pump. Once the pump is filled with the mixture, the priming valve is closed and the mixture is forced through the Interaction Chamber. The geometry of the Interaction Chamber produces powerful forces of sheer, impact and cavitation which reduce particle size. Inside the Interaction Chamber, the pressurized mixture is split into two streams and accelerated to extremely high velocities. The formed jets are then directed toward each other and collide in the interaction zone. The resulting product has very fine and uniform particle size.

The distribution of medicament particles formed by any of the above exemplary techniques has an effective average particle size of less than or about 2000 nm (2 μm), 1900 nm, 1800 nm, 1700 nm, 1600 nm, 1500 nm, 1400 nm, 1300 nm, 1200 nm, 1100 nm, 1000 nm (1 μm), 900 nm, 800 nm, 700 nm, 600 nm, 500 nm, 400 nm, 300 nm, 200 nm, 150 nm, 100 nm, 75 nm, and 50 nm (nm=nanometers or 10⁻⁹ m).

The distribution of medicament particles is also characterized by a D₉₀. The D₉₀ of the distribution of medicament particles according to an embodiment of the invention is less than or about 5000 nm, 4900 nm, 4800 nm, 4700 nm, 4600 nm, 4500 nm, 4400 nm, 4300 nm, 4200 nm, 4100 nm, 3000 nm, 3900 nm, 3800 nm, 3700 nm, 3600 nm, 3500 nm, 3400 nm, 3300 nm, 3200 nm, 3100 nm, 3000 nm 2900 nm, 2800 nm, 2700 nm, 2600 nm, 2500 nm, 2400 nm, 2300 nm, 2200 nm, 2150 nm, 2100 nm, 2075 nm, 2000 nm (2 μm), 1900 nm, 1800 nm, 1700 nm, 1600 nm, 1500 nm, 1400 nm, 1300 nm, 1200 nm, 1100 nm, 1000 nm (1 μm), 900 nm, 800 nm, 700 nm, 600 nm, 500 nm, 400 nm, 300 nm, 200 nm, 150 nm, 100 nm, 75 nm, and 50 nm.

The drug delivery composition comprises one or more semipermeable coatings that does not adversely affect the drug, animal body, or host. The semipermeable coating substantially prevents the passage of medicament particles out of the drug delivery composition, but allows dissolved medicaments to be release from within the composition. In an embodiment, the semipermeable coating is the outermost layer of the composition.

The semipermeable coating is present in the drug delivery composition in an amount that ranges from 1% to 50%, and an amount in between, for example, 1%, 3%, 5%, 7%, 9%, 10%, 11%, 12%, 13%, 14%, 15%, 16%, 17%, 18%, 19%, 20%, 22%, 25%, 30%, 35%, 40%, and 50% based upon the total weight of the drug delivery composition. The amount of semipermeable coating in the composition may also be expressed as a range between any of the above-listed individual percentages.

In certain embodiments, the semipermeable coating is a controlled-porosity microporous coating, one or more water-swellable polymers, or a combination thereof.

The controlled-porosity microporous coating comprises: (1) a polymer that is insoluble in the environment of use, (2) a pore forming additive soluble in the environment of use and dispersed throughout the microporous coating, and optionally, (3) other excipients. Suitable exemplary, controlled-porosity microporous coatings are described in WO/2001/032149 herein incorporated by reference.

The controlled-porosity microporous coating visually appears as a sponge-like structure composed of numerous open and closed cells that form a discontinuous interwoven network of void spaces when viewed with a scanning electron microscope. The physical characteristics of the controlled-porosity microporous coating, i.e., the network of open and closed cells, serve as both an entry point for environmental fluid and as an exit for dissolved medicament. The pores can be continuous pores that have an opening on both faces of the controlled-porosity microporous coating (i.e., the inner surface facing the center of the drug delivery composition and the exterior surface facing the environment of use). The pores may be interconnected through tortuous paths of regular and irregular shapes including curved, curved-linear, randomly oriented continuous pores, hindered connected pores and other porous paths discernible by microscopic examination. Generally, the controlled-porosity microporous coating is defined by the pore size, the number of pores, the tortuosity of the microporous path and the porosity which relates to the size and number of pores. The pore size of the controlled-porosity microporous coating is easily ascertained by measuring the observed pore diameter at the surface of the material under the electron microscope. Generally, materials possessing from about 5% to about 95% pores and having a pore size from about 10 angstroms to about 100 microns can be used. The controlled-porosity microporous coating, as constituted in the environment of use, has a small solute reflection coefficient, σ, and displays poor semipermeable characteristics when placed in a standard osmosis cell.

Exemplary polymers that are insoluble in the environment of use and comprise the controlled-porosity microporous coating include cellulosic polymers, methacrylates and phthalates.

More specifically, exemplary polymers include cellulose acetates having a degree of substitution, D.S., meaning the average number of hydroxyl groups on the anhydroglucose unit of the polymer replaced by a substituting group, up to 1 and acetyl content up to 21%; cellulose diacetate having a D.S. of 1 to 2 and an acetyl content of 21 to 35%; cellulose triacetate having a D. S. of 2 to 3 and an acetyl content of 35 and 44.8%; cellulose propionate having an acetyl content of 1.5 to 7% and a propionyl content of 39.2 and 45% and hydroxyl content of 2.8 to 5.4%; cellulose acetate butyrate having a D.S. of 1. 8, an acetyl content of 13 to 15% and a butyryl content of 34 to 39%; cellulose acetate having an acetyl content of 2 to 99.5%, a butyryl content of 17 to 53%, and a hydroxyl content of 0.5 to 4.7%; cellulose triacetylates having a D. S. of 2. 9 to 3 such as cellulose trivalerate, cellulose trilaurate, cellulose tripalmitate, cellulose trisuccinate, cellulose triheptylate, cellulose tricaprylate, cellulose trioctanoate, and cellulose tripropionate; cellulose diesters having a lower degree of substitution and prepared by the hydrolysis of the corresponding triester to yield cellulose diacylates having a D.S. of 2.2 to 2.6 such as cellulose dicapyrlate and cellulose dipentanate; and esters prepared from acyl anhydrides or acyl acids in an esterification reaction to yield esters containing different acyl groups attached to the same cellulose polymer such as cellulose acetate valerate, cellulose acetate succinate, cellulose propionate succinate, cellulose acetate octanoate, cellulose valerate palmitate, cellulose acetate palmitate and cellulose acetate heptanoate and the like.

Additional exemplary polymers include cellulose acetate acetoacetate, cellulose acetate chloroacetate, cellulose acetate furoate, dimethoxyethyl cellulose acetate, cellulose acetate carboxymethoxy-propionate, cellulose acetate benzoate, cellulose butyrate napthylate, methylcellulose acetate methylcyanoethyl cellulose, cellulose acetate methoxyacetate, cellulose acetate ethoxyacetate, cellulose acetate dimethylsulfamate, ethylcellulose, ethyl-cellulose dimethylsulfamate, cellulose acetate p-toluene sulfonate, cellulose acetate methylsulfonate, cellulose acetate dipropylsulfamate, cellulose acetate butylsulfonate, cellulose acetate laurate, cellulose stearate, cellulose acetate methylcarbamate, agar acetate, amylose triacetate beta glucan acetate, beta glucan triacetate, acetaldehyde dimethyl acetate, cellulose acetate ethyl carbamate, cellulose acetate phthalate, cellulose acetate dimethyl aminoacetate, cellulose acetate ethyl carbonate, poly (vinyl methyl)ether copolymers, cellulose acetate with acetylated hydroxy-ethyl cellulose hydroxylated ethylenevinylacetate, poly ortho esters, polyacetals, semipermeable polyglycolic or polyactic acid and derivatives thereof, selectively permeable associated polyelectrolytes, polymers of acrylic and methacrylic acid and esters thereof, film forming materials with a water sorption of one to fifty percent by weight at ambient temperatures with a presently preferred water sorption of less than thirty percent, acylated polysaccharides, acylated starches, aromatic nitrogen containing polymeric materials that exhibit permeability to aqueous fluids, membranes made from polymeric epoxides, copolymers of alkylene oxides and alkyl glycidyl ethers, polyurethanes, and the like. Admixtures of various polymers may also be used.

The polymers described are known to the art or they can be prepared according to the procedures in Encyclopedia of Polymer Science and Technology, Vol. 3, pages 325 to 354 and 459 and 549, published by Interscience Publishers, Inc., New York, in Handbook of Common Polymers by Scott, J. R. and Roff, W. J., 1971, published by CRC Press, Cleveland, Ohio; and in U.S. Pat. Nos. 3,133,132; 3,173,876; 3,276,586; 3,541,055; 3,541,006; and 3,546,142.

The pore forming additive defines the porosity of the controlled-released microporous coating. The porosity of the controlled-release microporous coating may be formed in situ, by the pore forming additive being removed by dissolving or leaching it to form the microporous coating during the operation of the system. The pores may also be formed prior to operation of the system by gas formation within curing polymer solutions which result in voids and pores in the final form of the coating. The pore forming additive can be a solid or a liquid.

An exemplary pore forming additive soluble in the environment of use, according to exemplary embodiments, is the pore forming additive sold under the tradename Opadry® by Colorcon, Inc. of West Point, Pa.

According to other embodiments, the pore forming additives include, but are not limited to, HPMC, PVP, polyhydric alcohols, or sugars.

Yet in other embodiments, the pore forming additive is an inorganic or organic compound. The pore forming additives suitable for the invention include a pore forming additives that can be extracted without any chemical change in the polymer. Solid additives include alkali metal salts such as sodium chloride, sodium bromide, potassium chloride, potassium sulfate, potassium phosphate, sodium benzoate, sodium acetate, sodium citrate, potassium nitrate and the like. The alkaline earth metal salts such as calcium chloride, calcium nitrate, and the like. The transition metal salts such as ferric chloride, ferrous sulfate, zinc sulfate, cupric chloride, and the like. Water may be used as the pore-former. These pore forming additives include organic compounds such as saccharides. The saccharides include the sugars sucrose, glucose, fructose, mannose, galactose, aldohexose, altrose, talose, lactose, monosaccharides, disaccharides, and water soluble polysaccharides. Also, sorbitol, manitol, organic aliphatic and aromatic ols, including diols and polyols, as exemplified by polyhydric alcohols, poly(alkylene glycols), polygylcols, alkylene glycols, poly(a-co)alkylenediols, esters or alkylene glycols poly vinylalcohol, poly vinyl pyrrolidone, and water soluble polymeric materials. Pores may also be formed in the microporous coating by the volatilization of components in a polymer solution or by chemical reactions in a polymer solution which evolves gases prior to application or during application of the solution to the cores mass resulting in the creation of polymer foams serving as the microporous coating of the invention. The pore forming additives are nontoxic, and on their removals, channels form that fill with fluid. In a preferred embodiment, the non-toxic pore forming additives are selected from the group consisting of inorganic and organic salts, carbohydrates, polyalkylene glycols, poly(a-co)alkylenediols, esters of alkylene glycols, and glycols that are used in a biological environment.

Processes for preparing microporous coatings are described in Synthetic Polymer Membranes, by R. E. Kesting, Chapters 4 and 5, 1971, published by McGraw Hill, Inc.; Chemical Reviews, Ultrafiltration, Vol. 18, pages 373 to 455, 1934; Polymer Eng. And Sci., Vol. 11, No. 4, pages 284-288, 1971; J. Appl. Poly. Sci., Vol. 15, pages 811 to 829, 1971; and in U.S. Pat. Nos. 3,565,259; 3,615,024 ; 3,751,536; 3,801,692; 3,852,224; and 3,849,528.

The percent by weight of pore forming additive in the controlled-porosity microporous coating is from about 0.5%, 0.75%, 1.0%, 1.3%, 1.5%, 1.7%, 1.9%, 2.0%, 2.5%, 3.0%, 3.5%, 4% , 4.5%, 5%, 6%, 7%, 8%, 9%, 10%, 12%, 13%, 15%, 17%, 19%, 21% , 22%, 24%, 26%, 28%, 30%, 32%, 34%, 36%, 38%, 41%, 43%, 45%, 47%, 49%, and 50%. The amount of pore forming additive in the composition may also be expressed as a range between any of the above-listed individual percentages.

In yet another embodiment of the invention, the semipermeable coating comprises one or more water-swellable polymers. The water-swellable polymers form a hydrophilic matrix that substantially prevents release of medicament particles, while simultaneously allowing passage of dissolved medicament into the environment of use. These polymers, when in contact with the environment of use, absorb the fluid and swell to form a viscous gel.

Exemplary water-swellable polymers include the Methocel™ methylcellulose and hypromellose systems of water-soluble cellulose ethers sold by The Dow Chemical Company of Midland, Mich., USA.

### Examples

The following examples are intended to illustrate various embodiments of the invention.

### Example 1

An exemplary drug delivery composition for a neutral compound according to the present invention comprises the following:

| Ingredient | Component | mg/dose |
|---|---|---|
| Sugar sphere | Inert core | 83.01 |
| Sodium | Solubilizing | 39.06 |
| Lauryl | agent | |
| Sulfate | | |
| Surelease ® | Water insoluble | 19.25 |
| | polymer | |
| Opadry ® | Pore former | 2.139 |
| Docusate | Stabilizer | 0.375 |
| sodium | | |
| Hypromellose | Stabilizer | 1.250 |
| Pearlitol ® | Dispersing aid | 5.000 |
| Active | Tacrolimus | 5.000 |
| ingredient | | |

and is manufactured as follows:

Approximately 1100 g to 2300 g of 20-30% w/w sodium lauryl sulfate (SLS) solution was sprayed on to 1000g of 30-35 mesh sugar spheres. The nanoparticulate tacrolimus was converted into a coating feed dispersion (CFD). The CFD comprised an aqueous colloidal dispersion of tacrolimus, additional stabilizers, and dispersing agent. Approximately 1200 g of 5% w/w of the coating feed dispersion was spayed onto the SLS coated beads. In a final step, a dispersion of approximately 1600g of 15% w/w water-insoluble polymer and pore former (90:10 water-insoluble polymer to pore former ratio) was applied onto 1500 g of CFD coated beads. The coated beads were cured for 3 hr in an oven.

### Example 2

Example 2 is a comparison between a drug delivery composition containing a solubilizing agent, a drug delivery composition that does not include a solubilizing agent, and a dosage form of the drug in nanoparticulate form without the solubilizing agent or semipermeable coating.

### Composition A: With Solubilizing Agent (Sodium Lauryl Sulfate)

| Ingredient | Component | mg/dose |
|---|---|---|
| Sugar sphere | Inert core | 83.00 |
| Sodium | Solubilizing | 39.10 |
| Lauryl | agent | |
| Sulfate | | |
| Surelease ® | Water-insoluble | 12.00 |
| | polymer | |
| Opadry ® | Pore former | 1.34 |
| Docusate | Surface | 0.38 |
| sodium | Stabilizer | |
| Hypromellose | Surface | 1.25 |
| | Stabilizer | |
| Pearlitol ® | Dispersing aid | 5.00 |
| Tacrolimus | Medicament | 5.00 |

Composition B: No Solubilizing Agent (No Sodium Lauryl Sulfate)

| Ingredient | Component | mg/dose |
|---|---|---|
| Sugar sphere | Inert core | 122.00 |
| Sodium | Solubilizing | — |
| Lauryl | agent | |
| Sulfate | | |
| Surelease ® | Water-insoluble | 12.00 |
| | polymer | |
| Opadry ® | Pore former | 1.34 |
| Docusate | Surface | 0.38 |
| sodium | Stabilizer | |
| Hypromellose | Surface | 1.25 |
| | Stabilizer | |
| Pearlitol ® | Dispersing aid | 5.00 |
| Tacolimus | Medicament | 5.00 |

### Composition C: No Solubilizing Agent and No Semipermeable Coating

| Ingredient | Component | mg/dose |
|---|---|---|
| Sugar sphere | Inert core | 123.00 |
| Sodium | Solubilizing agent | — |
| Lauryl | | |
| Sulfate | | |
| Surelease ® | Water-insoluble | — |
| | polymer | |
| Opadry ® | Pore former | — |
| Docusate | Surface Stabilizer | 0.38 |
| sodium | | |
| Hypromellose | Surface Stabilizer | 1.25 |
| Pearlitol ® | Dispersing aid | 5.00 |
| Tacrolimus | Medicament | 5.00 |

Compositions A, B and C were manufactured as set forth in Example 1.

Compositions A, B and C differed in their formulations; composition A included a solubilizing agent while composition B and C did not; both compositions A and B included a 10% semipermeable coating consisting of 90% water-insoluble polymer and 10% pore former. Composition C did not include a semipermeable coating.

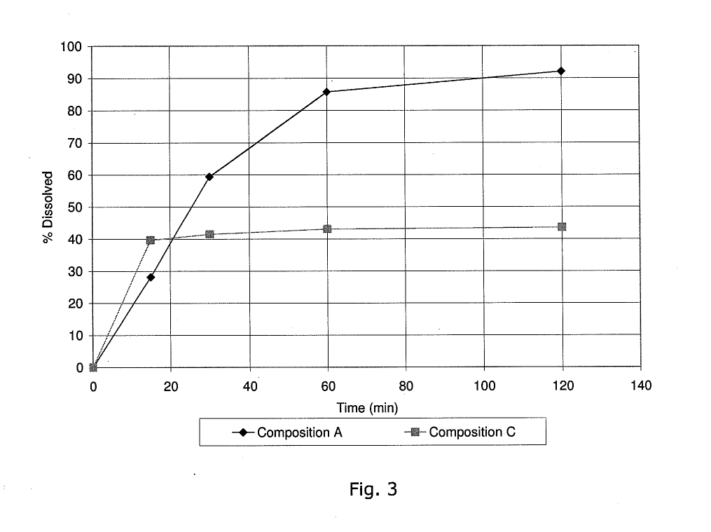

Compositions A, B, and C were placed in 0.005% HPC, pH 4.5 according to USP <711>, apparatus I (2009), Baskets at 100 rpm (the dissolution vehicle). As an example of the different release profiles for the three compositions, the amount of medicament released from composition A was 92.07% at 120 min. The amount of medicament released from composition B was less than 10% at 360 min (excluded from graph due to scale). The amount of medicament released from Composition C was 43.55% at 120 min. For reference, the native solubility of tacrolimus in this dissolution vehicle equates to approximately 43% dissolved. A plot of the percentage of drug dissolved over time for compositions A and B is shown in FIG. 3.

### Example 3

Example 3 represents a pharmacokinetic comparison of the medicament tacrolimus formulated in the drug delivery composition of the invention versus a nanoparticulate tacrolimus formulation.

The reference compositions described as Composition C in Example 2 and the drug delivery composition described in Example 1 (referred to herein as “Composition D”) were tested for pharmacokinetic properties.

The pharmacokinetics of the Composition D and Composition C were evaluated following oral crossover administration to male beagle dogs. Prior to dosing, the animals were fasted overnight. A pre-study health check was performed and a predose blood sample was taken. Blood samples were taken at 5, 10, 20, 30, 45, 60, 90 minutes and 2, 3, 4, 6, 8, 12, 24 and 48 hours post dose. Whole blood samples were frozen at −70° C. until transferred to the bioanalytical lab for tacrolimus concentration analysis. Plasma concentrations of tacrolimus were measured by liquid chromatography-mass spectrometry (LC-MS) with a quantitation limit of 0.100 ng/mL. A pharmacokinetic analysis was performed using a noncompartmental analysis using WinNonlin® software sold by Pharsight®, a Mountain View, California company.

The table below describes a comparison of the critical PK parameters for this evaluation—the treatment-to-reference ratios for Cₘₐₓ and AUCₗₐₛₜ. In this comparison, Composition C is the reference product (R) and Composition D is the treatment product (T). By comparing the treatment-to-reference ratios for Cₘₐₓ and AUCₗₐₛₜ, it is clear that Composition D resulted in a higher Cₘₐₓ and greater AUCₗₐₛₜ.

| | Ratio | Ratio |
|---|---|---|
| | Cmax | AUClast |
| Subject | (T/R) | (T/R) |
| 1001 | 2.23 | 1.99 |
| 2001 | 1.74 | 2.17 |
| 3001 | 0.32 | 0.31 |
| 4001 | 0.82 | 1.20 |
| 5001 | 0.67 | 1.19 |
| 6001 | 3.34 | 1.36 |
| Mean | 1.52 | 1.37 |
| SD | 1.14 | 0.66 |

### Example 4

Example 4 demonstrates the amount of dissolved drug in the fluid environment using an exemplary drug delivery composition comprising a weakly basic compound, clozapine, and a pH-modulating agent when compared to a clozapine control formulation, e.g., commercially available immediate-release clozapine tablets.

The established intrinsic solubility of clozapine is 0.016 mg/mL. The pka values for clozapine are 3.98 and 7.62. The theoretically calculated saturation solubility based upon these known values for of bulk clozapine API at pH 6.8 was estimated at 0.12 mg/mL.

The concentration of clozapine delivered from the drug delivery composition of the invention to a fluid environment was determined in 0.1M sodium phosphate buffer, pH 6.8 at 37° C., which is representative of the fluid environment of the human small intestine. The formulation of the drug delivery composition of this Example 4 is described in the table below.

| Ingredient | Component |
|---|---|
| Clozapine | Medicament |
| Hypromellose | Surface Stabilizer |
| Docusate | Surface Stabilizer |
| Sodium | |
| Perlitol ® | Dispersing agent |
| (mannitol) | |
| Sodium Lauryl | Surface-active |
| Sulfate | agent |
| Sugar Spheres | Inert core |
| Tartaric acid | pH-modulating agent |
| Opadry ® (pore | Pore former |
| former) | |
| Surelease ® | Water-insoluble |
| | polymer |

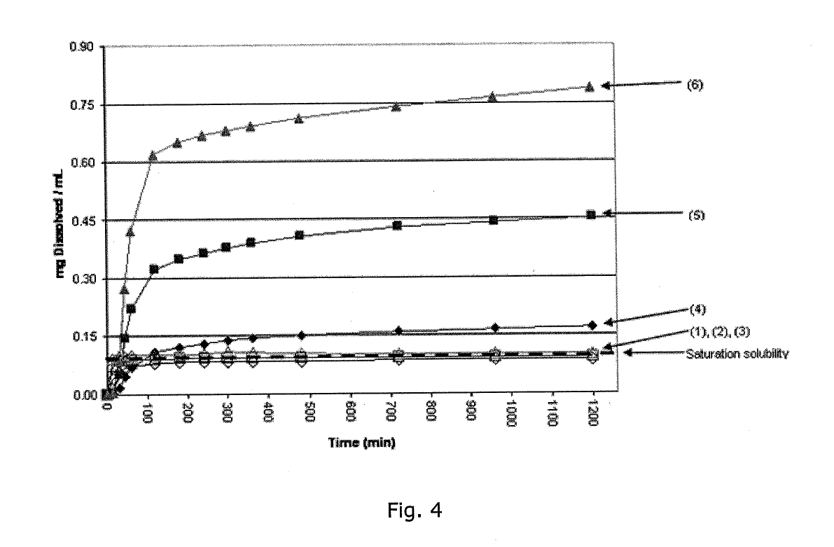

Three separate quantities of the above composition were studied corresponding to 200 mg, 600 mg and 1200 mg of clozapine. These compositions were placed in 1000 mL of 0.1M sodium phosphate, pH 6.8 according to USP <711>, apparatus II (2009), paddles at 75 rpm. Control experiments were performed using 200 mg, 600 mg and 1200 mg of clozapine in the form of immediate-release tablets. Comparative dissolution results of the composition of the invention and the control clozapine formulation are set forth in the table below. A graphical representation of this data is expressed in FIG. 4. Lines (1), (2) and (3) represent the profiles obtained for the 200 mg, 600 mg and 1200 mg samples of clozapine control tablets. Line (4) represents the profile obtained for nominal 200 mg of clozapine. Line (5) represents the profile obtained for nominal 600 mg of clozapine, and Line (6) represents the profile obtained for nominal 1200 mg of clozapine.

| | | Experimentally | Ratio of |
|---|---|---|---|
| | | determined | clozapine |
| | Experimentally | concentration | concentration |
| | determined | as a | achieved with |
| | concentration | percentage of | composition of |
| | in mg/mL of | the | the invention to |
| | clozapine at T = | anticipated | the concentration |
| | 20 hours in | clozapine | with equivalent |
| | pH 6.8, 0.1M | saturation | amount of |
| Sample | sodium | solubility at | clozapine control |
| description | phosphate | pH 6.8 | formulation |
| 200 mg clozapine | 0.087 | 71.3 | — |
| (control) | | | |
| 600 mg clozapine | 0.094 | 76.9 | — |
| (control) | | | |
| 1200 mg | 0.102 | 83.9 | — |
| clozapine | | | |
| (control) | | | |
| 204 mg | 0.171 | 140 | 1.96 |
| clozapine* | | | |
| 624 mg | 0.453 | 371 | 4.82 |
| clozapine* | | | |
| 1203 mg | 0.787 | 645 | 7.72 |
| clozapine* | | | |

*Clozapine was formulated into the drug delivery composition of the invention.

After 20 hours the measured concentration of the control clozapine formulation in the environmental fluid approached, but did not reach, the anticipated saturation solubility for the 200 mg, 600 mg, and 1200 mg sample sizes. Rather, the 600 mg and 1200 mg sample sizes of control clozapine formulation achieved values of 0.094 mg/mL and 0.102 mg/mL, respectively. The concentration of clozapine delivered from the drug delivery composition of the invention to the pH 6.8 sodium phosphate buffer far exceeded that achieved from the experiments using an equivalent quantity of the control clozapine tablet formulation.

For the nominal 200 mg sample the drug delivery composition of the invention delivered a clozapine concentration of 0.171 mg/mL (140% of the theoretical saturation solubility) or a factor of 1.96 times the concentration achieved with an equivalent amount of control clozapine tablet formulation.

For the nominal 600 mg sample the drug delivery composition delivered a concentration of clozapine at 0.453 mg/mL (371% of the theoretical saturation solubility) or a factor of 4.82 times the concentration achieved with an equivalent amount of the control clozapine tablet formulation.

For the nominal 1200 mg sample the drug delivery composition delivered a concentration of clozapine at 0.787 mg/mL (645% of the anticipated saturation solubility) or a factor of 7.69 times the concentration achieved with an equivalent amount of the control clozapine tablet formulation.

### Example 5

A diagnostic formulation model system was established to support the drug delivery compositions of the invention. This model system encompassed a semipermeable membrane, medicament particles and a solubilizing agent. The model system was designed with multiple features to provide flexibility to address a wide variety of formulation variables and different in vitro release experiments that may be required to support the drug delivery composition of the invention.

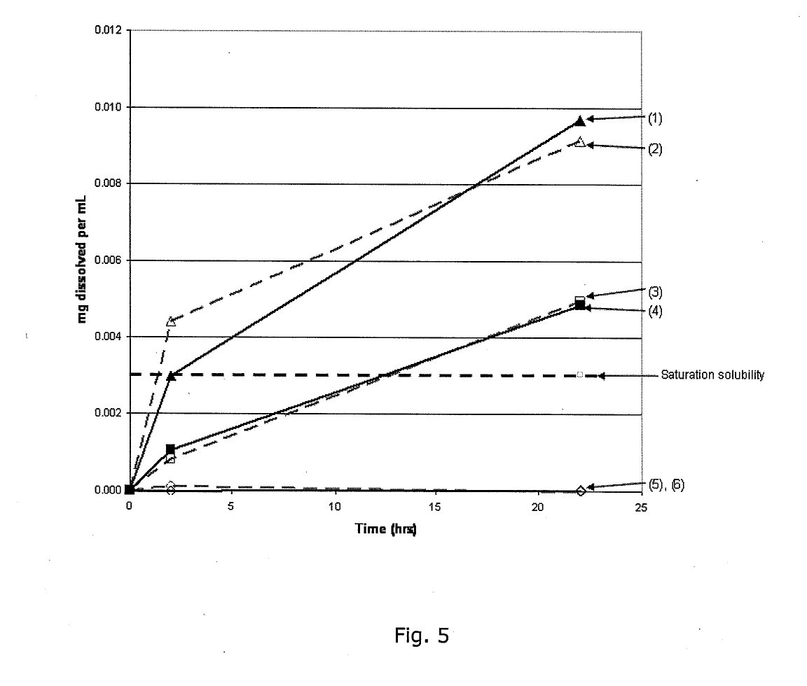

Shown in FIG. 5 is a dissolution profile for a weakly basic compound, dipyridamole, with a weak acid pH modulating agent using the model system. The plot shows the mg per mL dissolved over dissolution time. Line (1) represents the dissolution profile of non-nanoparticulate API of dipryidamole with an acid pH modulating agent, L2. Line (2) represents the dissolution profile of nanoparticulate API of dipryidamole with acid pH modulating agent, L2. Line (3) represents the dissolution profile of non-nanoparticulate API of dipryidamole without a pH modulating agent, L1. Line (4) represents the dissolution profile of a nanoparticulate medicament form of dipryidamole with acid pH modulating agent, L1. Line (5) represents the dissolution profile of a nanoparticulate medicament form of dipryidamole without an acid pH modulating agent. And line (6) represents the dissolution profile of a bulk medicament form of dipryidamole without an acid pH modulating agent.

### Example 6

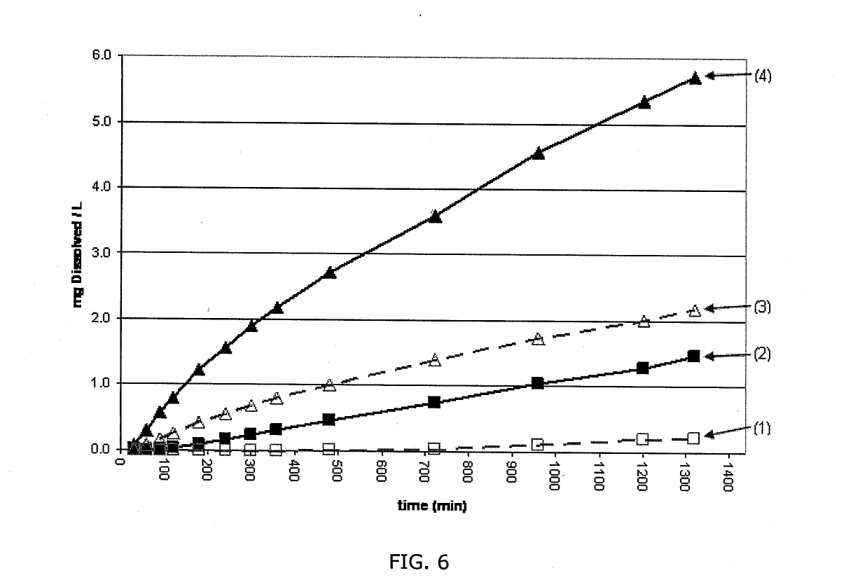

In this example, a model system in accordance with Example 5 comprising a basic drug, carvedilol, and a suitable weak acid pH modulating agent was studied. FIG. 6 is the profile plot of mg per mL dissolved over dissolution time.

Line (1) represents the dissolution profile of non-nanoparticulate API form of carvedilol without an acid pH modulating agent. Line (2) represents the dissolution profile of a nanoparticulate medicament form of carvedilol without an acid pH modulating agent. Line (3) represents the dissolution profile of a non-nanoparticulate API form of carvedilol with an acid pH modulating agent. And line (4) represents the dissolution profile of a nanoparticulate API form of carvedilol with an acid pH modulating agent.

### Example 7

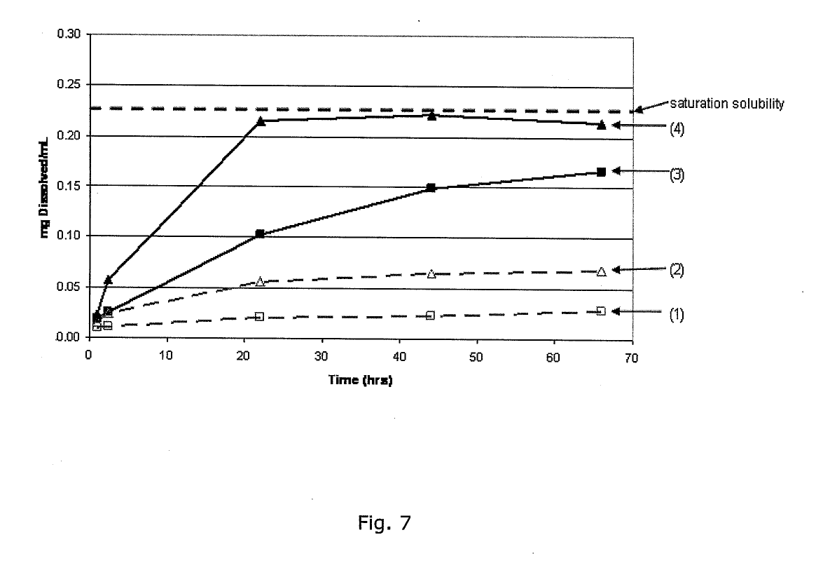

In this example, a surrogate system in accordance with Example 5 utilizing a weakly acidic drug, vorinostat, and a weak base pH modulating agent was studied. FIG. 7 is the dissolution profile plot of mg per mL dissolved over dissolution time.

Line (1) represents the dissolution profile of a non-nanoparticulate API form of vorinostat without a weak base pH modulating agent. Line (2) represents the dissolution profile of non-nanoparticulate API form of vorinostat with the weak base pH modulating agent. Line (3) represents the dissolution profile of a nanoparticulate API form of vorinostat without a weak base pH modulating agent. And line (4) represents the dissolution profile of a nanoparticulate API form of vorinostat with a weak base pH modulating agent.

What is claimed:

1. A composition comprising:
a semipermeable coating;
particles of a medicament having an effective average particle size of less than or about 2 μm and a surface stabilizer adsorbed on the surface of the medicament particles; and
a solubilizing agent.

2. The composition of claim 1, wherein the semipermeable coating is selected from the group consisting of a controlled-porosity microporous coating, a water swellable coating, and mixtures and combinations thereof.

3. The composition of claim 1, wherein the semipermeable coating is a controlled-porosity microporous coating comprising a polymer that is insoluble in an environment of use and a pore forming additive that is soluble in the environment of use.

4. The composition of claim 3, wherein the polymer is selected from the group consisting of cellulosic polymers, methacrylates and phthalates, and
wherein the pore forming additive is selected from the group consisting of HPMC, PVP, polyhydric alcohols, and sugars.

5. The composition of claim 3, wherein the percent by weight of pore forming additive in the controlled-porosity microporous coating is selected from the group consisting of 0.5%. 1.0%, 1.5%, 2.0%, 2.5%, 3.0%, 3.5%, 4%, 4.5%, 5%, 6%, 7%, 8%, 9%, 10%, 12%, 13%, 15%, 17%, 19%, 21%, 22%, 24%, 26%, 28%,) 30%, 32%, 34%, 36%, 38%, 41%, 43%, 45%, 47%, 49%, and 50%.

6. The composition of claim 1, wherein the medicament is selected from a class of medicaments selected from the group consisting of abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents, antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, ant igonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, COX-I and COX II inhibitors, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H2 receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, NSAIDS, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors.

7. The composition of claim 1, wherein the medicament is poorly soluble in an environment of use.

8. The composition of claim 1, wherein the effective average particle size is selected from the group consisting of less than or about 1900 nm, 1800 nm, 1700 nm, 1600 nm, 1500 nm, 1400 nm, 1300 nm, 1200 nm, 1100 nm, 1000 nm (1 μm), 900 nm, 800 nm, 700 nm, 600 nm, 500 nm, 400 nm, 300 nm, 200 nm, 150 nm, 100 nm, 75 nm, and 50 nm.

9. The composition of claim 1, wherein the particles of the medicament have a D₉₀ selected from the group consisting of less than or about 5000 nm, 4900 nm, 4800 nm, 4700 nm, 4600 nm, 4500 nm, 4400 nm, 4300 nm, 4200 nm, 4100 nm, 3000 nm, 3900 nm, 3800 nm, 3700 nm, 3600 nm, 3500 nm, 3400 nm, 3300 nm, 3200 nm, 3100 nm, 3000 nm 2900 nm, 2800 nm, 2700 nm, 2600 nm, 2500 nm, 2400 nm, 2300 nm, 2200 nm, 2150 nm, 2100 nm, 2075 nm, and 2000 nm.

10. The composition of claim 1, wherein the surface stabilizer is selected from the group consisting of hydroxypropyl methylcellulose (HPMC), dioctyl sodium sulfosuccinate (DOSS), sodium lauryl sulfate (SLS), hydroxypropyl cellulose, polyvinylpyrrolidone, sodium deoxycholate, block copolymers based on ethylene oxide and propylene oxide, copolymers of vinylpyrrolidone and vinyl acetate, lecithin, polyoxyethylene sorbitan fatty acid esters, albumin, lysozyme, gelatin, macrogol 15 hydroxystearate, tyloxapol, and polyethoxylated castor oil.

11. The composition of claim 1, wherein the solubilizing agent is of a type and present in an amount sufficient to dissolve the medicament particles within the composition prior to delivery of the medicament to an environment of use.

12. The composition of claim 11, wherein the solubilizing agent is a surface-active agent or a pH-modulating agent.

13. The composition of claim 11, wherein the surface-active agent is selected from the group consisting of anionic, cationic, zwitterionic and nonionic surface-active agents.

14. The composition of claim 12, wherein the solubilizing agent is a pH-modulating agent and, when exposed to fluid of the environment of use, modifies the pH environment within the composition to favor an ionized form of the medicament.

15. The composition of claim 12, wherein the solubilizing agent is a pH-modulating agent selected from a weak acid or a weak base.

16. The composition of claim 15, wherein the weak acid is selected from the group consisting of adipic acid, ascorbic acid, citric acid, fumaric acid, gallic acid, glutaric acid, lactic acid, malic acid, maleic acid, succinic acid, tartaric acid and mixtures and combinations thereof.

17. The composition of claim 15, wherein the weak base is selected from the group consisting of arginine, lysine, tromethamine (TRIS), meglumine, diethanolamine, triethanolamine, conjugate bases of pharmaceutically acceptable weak acids, and mixtures and combinations thereof.

18. The composition of claim 17, wherein the conjugate bases of pharmaceutically acceptable weak acids are selected from the group consisting of sodium carbonate, sodium phosphate, calcium phosphate, trisodium citrate, and sodium ascorbate and mixtures or combinations thereof.

19. The composition of claim 1, wherein the drug delivery composition delivers to an environment of use a solution of medicament at a concentration that is higher than that defined by the native solubility of the medicament in the environment of use.

20. The composition of claim 19, wherein the concentration of is 5%, 10%, 20%, 30%, 40%, 50%, 60%, 70%, 80%, 90% 100%, 110%, 120%, 130%, 140%, 150%, 160%, 170%, 180%, 190%, 200%, 210%, 220%, 230%, 240%, 250%, 260%, 270%, 280%, 290%, 300, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 700%, 800% or 1000% higher than that defined by the native solubility of the medicament in the environment of use.
